(19) United States
(12) Patent Application Publication
(10) Pub. No.: US 2009/0171270 A1
(43) Pub. Date: Jul. 2, 2009

(54) ATTACHMENT FOR A MILK PUMP

(76) Inventor: Peter Röhrig, Wien (AT)

Correspondence Address: LADAS & PARRY LLP, 224 SOUTH MICHIGAN AVENUE, SUITE 1600, CHICAGO, IL, 60604, US

(21) Appl. No.: 12/161,987

(22) PCT Filed: Jan. 24, 2007
(86) PCT No.: PCT/AT2007/000027

§ 371 Date: Jul. 24, 2008

(30) Foreign Application Priority Data
Jan. 24, 2006 (AT) A 107/2006

(51) Int. Cl. A61M 1/06; A61M 1/00

(52) U.S. Cl. 604/74; 604/316

(57) ABSTRACT

An attachment for a milk pump, including a one-walled, dimensionally stable positioning section which tapers in tunnel-shaped manner from the positioning opening to the securing section for connecting to the milk pump. The positioning section and the securing section are connected together by an articulated connection such that the longitudinal axis of the tunnel-shaped positioning section can be adjusted to an angle (alpha) which deviates by 180° in relation to the longitudinal axis of the securing section.

The invention relates to an attachment for a milk pump, including a single-walled, substantially dimensionally stable application portion tapering in a funnel-shaped manner from the application opening to a fixing portion provided for connection with the milk pump.

These days, the pumping of breast milk by the aid of a milk pump is common and done for various reasons. On the one hand, to eliminate inconveniences for the mother when her breasts are filled with milk and sometimes even cause pain. On the other hand, a mother can take her milk at any time by the aid of a milk pump for somebody else to feed her baby with the breast milk a later time. In addition, the use of a breast pump enables a mother suffering from acute breast inflammation or sore nipples to feed breast milk nevertheless. Besides, breast milk is the best form of nutrition for prematurely born babies, increasing the chance of survival of premature babies.

From GB 2 380 411, a milk pump is already known, in which an attachment having a substantially dimensionally stable application portion is provided, whose longitudinal axis is angularly arranged relative to the longitudinal axis of a fixing portion. The fixing portion in that case is rotationally received in a suction socket of the milk pump in such a manner as to achieve a change in the spatial orientation of the application opening by rotating the attachment within the suction socket of the milk pump. It is, however, disadvantageous that an adaptation of the orientation of the application opening is only difficult to perform during the pumping procedure.

Furthermore, a two-part attachment for a milk pump is known from US 2005/0256449 A, wherein an elastic inner wall is provided to stimulate the mother's breast. In addition, a separate external, funnel-shaped attachment is provided, which may comprise valve openings to enable a pressure compensation of the elastic inner wall.

From EP 1 034 807 A, a further two-part attachment is known, in which an inner elastomer layer is provided, which is surrounded by a dimensionally stable outer shell. The shell may be articulately received in a screw cap of the milk pump. There, it is particularly disadvantageous that cleaning is cumbersome because of the hard outer part and the soft insert.

It is the object of the present invention to provide an attachment for a milk pump, by which the ease of use of the attachment and milk pump in respect to an individual adaptation of the orientation of the application opening is enhanced and cleaning expenditures are kept low.

In accordance with the invention, the milk pump attachment of the initially defined kind is characterized in that the application portion and the fixing portion are interconnected by an articulated connection such that the longitudinal axis of the funnel-shaped application portion is adjustable relative to a longitudinal axis of the fixing portion at an angle deviating from 180°. By providing an articulated connection between the application portion and the fixing portion, an adaptation of the orientation of an application opening relative to the milk pump, or a pump lever, can be achieved in a simple manner even during the pumping of breast milk.

An adjustment of the angular arrangement between the application portion and the fixing portion can be achieved in a simple manner if the application portion and the fixing portion are interconnected by a universal-joint connection. In a constructionally simple manner, it is, moreover, also possible to achieve a pivoting movement of the single-walled application portion relative to the fixing portion, if the application portion and the fixing portion are interconnected by a flexible connecting portion.

In order to ensure the simple adjustment of the orientation of the two longitudinal axes of the application portion and the fixing portion relative to each other with the attachment advantageously retaining its angular shape, it is provided that the flexible connecting portion is folded like bellows or corrugated.

In order to achieve the desired dimensional stability of the application portion, on the one hand, and the flexibility of the connecting portion and/or fixing portion, on the other hand, it is advantageous if the attachment is made of at least two materials of different hardnesses.

In this respect, it is beneficial if a thermoplastic elastomer, silicone or rubber is provided as the softer material. In order to achieve the desired flexibility, it is, in particular, advantageous if the softer material has a Shore hardness A of between 20 and 70, preferably between 30 and 40.

By contrast, in order to ensure the dimensional stability of the application portion, it is advantageous if polypropylene, polycarbonate or another thermoplastic material is provided as the harder material. If the harder plastic has a Rockwell hardness R of between 80 and 120, preferably substantially 105, it will, in particular, be feasible to suck the breast milk from the mother's breast by the applied pumping pressure while, at the same time, applying a pulsating pressure to the breast via the application portion in a manner similar to the palate of an infant.

In order to enable in a simple manner a change in the orientation of the application opening while, at the same time, achieve a reliable fixation of the application portion as well as a suitable transmission of the pumping pressure to the mother's breast, it is advantageous, if the flexible connecting portion comprises at least a further plastic material differing from that of the application portion and the fixing portion. The portions made of different materials may be connected by material overlapping, by adhesion or by a mechanical connection between the materials, in particular by form-fit or friction-fit engagement, for instance in the form of perforated and/or corrugated regions.

A simple connection of the hard and soft materials is, in particular, provided if at least one web connecting the application portion and the fixing portion is provided in the flexible connecting portion, said web being made of the same plastic as the application and fixing portions. In order to provide a pressure-tight cover in the region of the connecting portion, it is beneficial if a softer plastic is sprayed on the web in the region of the connecting portion. In order to enhance the flexibility of the connecting web and for the purpose of providing an efficient connection between the web and the sprayed-on plastic, it is advantageous if the web is ribbed or corrugated.

If the flexible connecting portion is comprised of softer plastic than the application portion and the fixing portion, the fixing portion, which is comprised of a soft and hence elastic plastic, can be put over a suction socket of the mother's breast for connection with the same.

Likewise, it is feasible to achieve a dimensional stability of the attachment and, in particular, of the application portion as well as the desired flexibility of the connecting portion, if the attachment comprises struts of the harder material and a closed cover of the softer material. In this respect, it is, in particular, advantageous if the attachment comprises struts extending in the direction of the longitudinal axis, wherein it is advantageous for the achievement of a dimensional stability, if the attachment comprises circular struts arranged perpendicularly to the longitudinal axis, in particular, in the region of the application portion.

With a view to ensuring a reliable connection between the two materials of different hardnesses, it is advantageous if the attachment is a one-piece two-component injection molded part.

In order to enable the simple insertion of the fixing portion in a conventional cylindrical or conical suction socket of a milk pump, it is advantageous if the fixing portion is conically or cylindrically designed.

In order to achieve an ergonomically favorable adaptation of the orientation of the application opening relative to the pump lever, it is beneficial if the longitudinal axis of the application portion is adjustable relative to the longitudinal axis of the fixing portion at an angle of between 150° and 170°, preferably substantially 165°.

In addition, the invention relates to a milk pump including a pumping device having a suction socket in communication with a funnel-shaped application portion of an attachment to be applied to the mother's breast, wherein the spatial orientation of a longitudinal axis of the funnel-shaped application portion relative to a longitudinal axis of the suction socket is adjustable, characterized in that an attachment according to any one of claims 1 to 22 is provided.

In the following, the invention will be explained in even more detail by way of the preferred exemplary embodiments illustrated in the drawings, to which it is, however, not to be restricted. In detail, in the drawing:

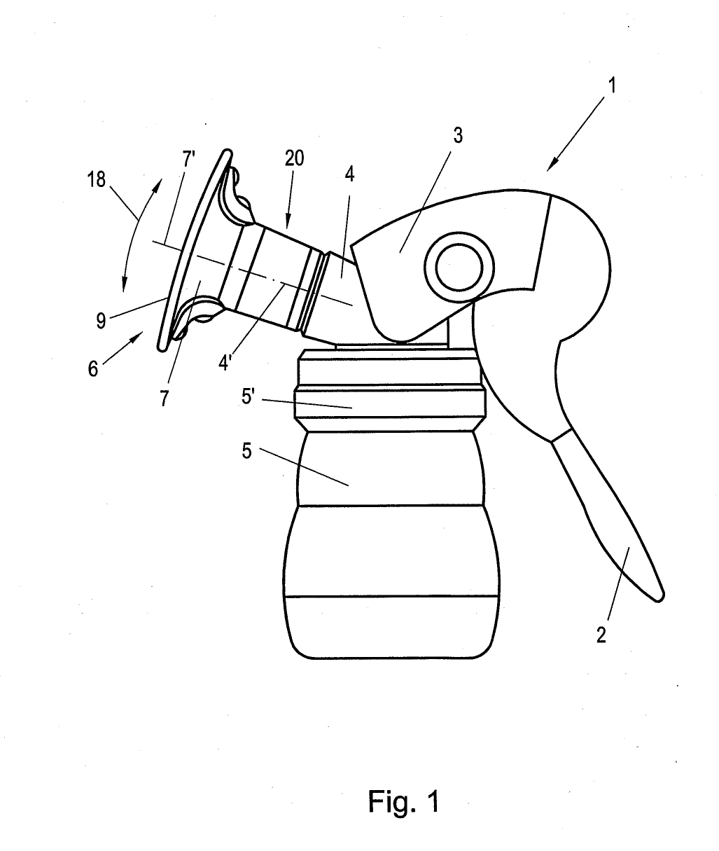

FIG. 1 is a view of a milk pump with an attachment;

FIG. 2 is a view of an attachment in which the application portion and the fixing portion are interconnected by a universal joint;

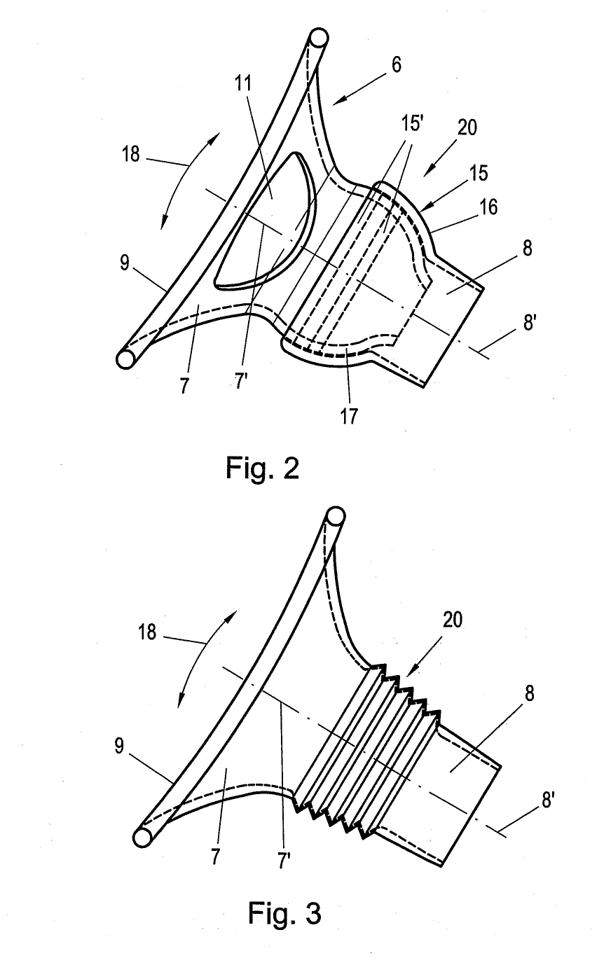

FIG. 3 is a view of an attachment in which the application portion and the fixing portion are interconnected by an articulated connecting portion folded like bellows;

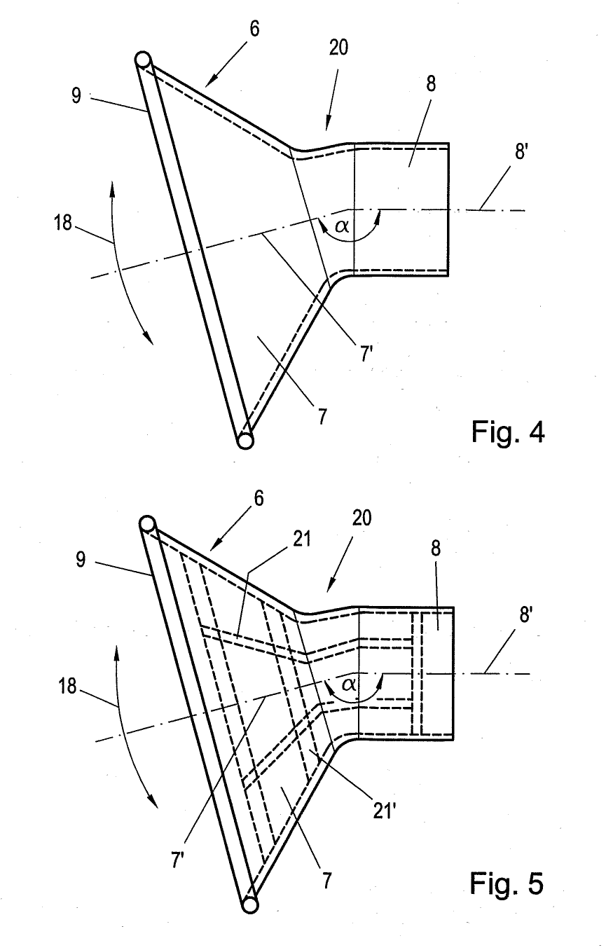

FIG. 4 is a view of an attachment in which the application portion and the fixing portion are interconnected by a connecting portion of soft plastic;

FIG. 5 is a view of an attachment made of soft plastic and reinforced by struts of harder plastic;

FIG. 6 is a view of an attachment having an application portion of harder plastic and a connecting and a fixing portion of softer plastic;

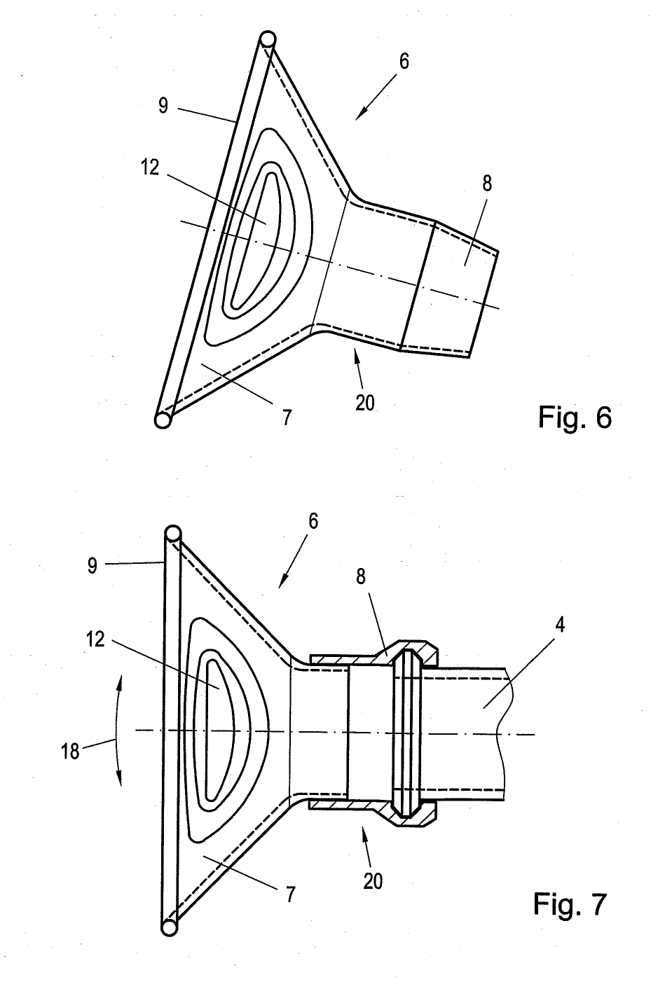

FIG. 7 is a view of an attachment similar to that of FIG. 6 in a state put on the socket of a milk pump;

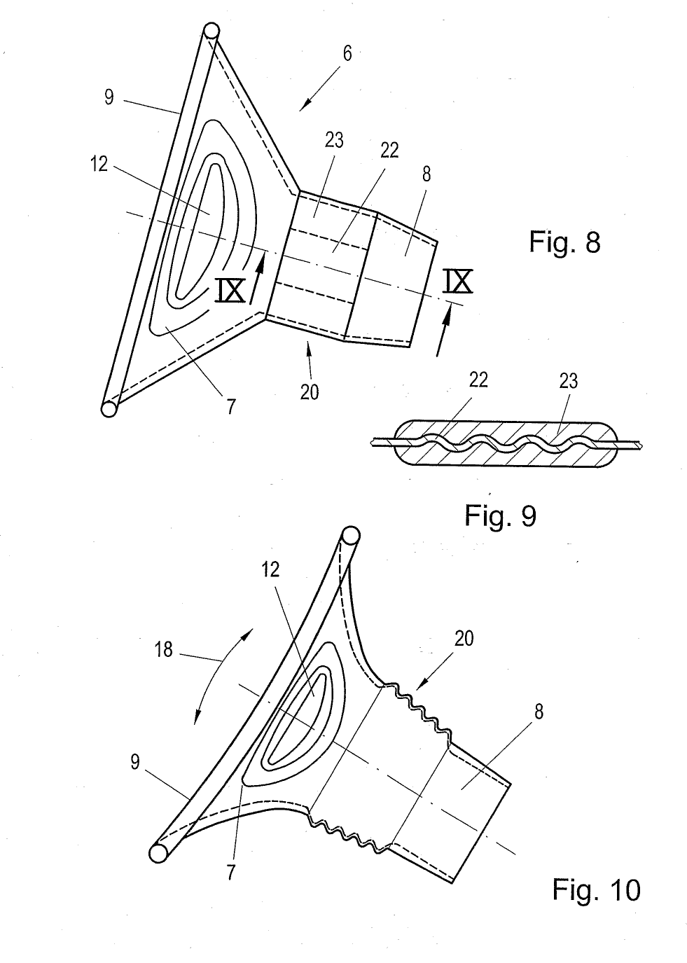

FIG. 8 is a view of an attachment including a connection web hard plastic in the connecting portion;

FIG. 9 is a section along line IX-IX;

FIG. 10 is a view of a suction socket similar to that of FIG. 3, yet with a bellows-shaped connecting portion of soft plastic.

FIG. 1 depicts a manually operable milk pump 1 including a pumping device 3 having a pump lever 2 and screwed on a reception container 5 for the pumped-off breast milk via a connection cap 5′. An attachment 6 is fastened in a tubular socket 4 of the pumping device 3. The attachment 6 has a funnel-shaped application portion 7 with a longitudinal axis 7′ and a connecting portion 20 adjoining the application portion 7 and adjoined by a fixing portion 8 (cf. FIG. 3). The connecting portion 20 is designed to be flexible so as to enable pivoting in any direction as indicated by arrow 18.

In the attachment 6 represented in FIG. 2, a universal joint 15 is provided in the connecting portion 20 so as to enable the adjustment of the longitudinal axis 7′ of the funnel-shaped application portion 7 relative to the rotational axis 8′ of the fixing portion 8. The universal joint 15 in this case comprises a substantially semi-spherical receiving portion 16 connected with the fixing portion 8 and in which an appropriate end portion 17 adjoining the application portion 7 is pivotally received. In order to avoid the suction of air into the joint gap between portions 16 and 17, two sealing rings 15′ are arranged between the two portions 16, 17. It is thus feasible in a simple manner by the aid of the universal joint 15 to change the orientation of the application opening 9 in the sense of arrow 18, the universal joint advantageously allowing for a rotation about 360°. The application portion, moreover, comprises two relatively diametrically arranged perforations 11 to enable the application of a manual massage pressure in the attached state of the attachment 6.

FIG. 3 depicts a further exemplary embodiment of the attachment 6, in which the application portion 7 is arranged to be pivotable relative to the fixing portion 8 in a manner that the longitudinal axis 7′ of the application portion can be arranged in an angular position deviating from 180° relative to the rotational axis 8′ of the fixing portion 8. To this end, an articulated connecting portion 20 is provided, in which the plastic material is folded like bellows so as to readily enable the achievement of a pivotal movement of the application portion 7 relative to the fixing portion 8.

Furthermore, FIG. 4 illustrates an exemplary embodiment in which the attachment 6 comprises an articulated connecting portion 20 between the fixing portion 8 and the application portion 7, said connecting portion 20 being made of a softer material than the application portion 7 and the fixing portion 8 such that—as shown in FIG. 4—pivoting of the application portion 7, and the orientation of the application opening 9, are feasible due to the elasticity of the soft material in the connecting portion 20. In the pivoted state illustrated in FIGS. 4 and 5, the longitudinal axes 7′, 8′ of the application and fixing portions 7, 8 thus enclose an angle α deviating from 180°.

FIG. 5 depicts a further exemplary embodiment of the attachment 6, which is almost entirely made of a comparatively soft material such as, e.g., elastomer, silicone or rubber, having a Shore hardness of between 20 and 70, advantageously between 30 and 40. In order to ensure the dimensional stability of the attachment 6, the attachment 6 comprises struts 21, 21′ extending both substantially in the sense of the longitudinal extension of the attachment 6 and in a plane arranged substantially perpendicular thereto. This enables in a simple manner (except in the regions where the comparatively stiff struts are arranged) to largely simulate, by manual massage, the natural “stripping” of a baby during the food ingestion via its mother's breast in addition to the negative pressure applied by the milk pump.

FIG. 6 illustrates an attachment 6 in which the adaptation portion 7 is made of harder plastic, namely polypropylene (PP) and the connecting portion 20 as well as the fixing portion 8 are comprised of softer plastic, namely a thermoplastic elastomer (TPE). Depending on the diameter of the fixing portion 8 and the associated socket 4 of the milk pump 1, the fixing portion 8 comprised of an elastic TPE material can be pulled over the socket 4 for the purpose of connection with the milk pump 1 (cf. FIG. 7). Alternatively, plugging into a socket 4 having a slightly larger diameter is, however, also feasible. The connection between the comparatively hard PP material and the comparatively soft TPE material in this case may be realized via overlapping material regions (cf. FIG. 7) as well as by adhesion or even by a mechanical material connection. In addition, diametrically opposite massage areas 12 are provided, which are likewise made of the softer material for the application of a manual massage pressure.

In FIGS. 8 and 9, a further exemplary embodiment of the attachment 6 is illustrated, wherein, in this case, a base body forming the application portion 7 as well as the fixing portion 8 and made of harder plastic, preferably PP, is provided, which, in addition, comprises at least one connecting web 22. Since the web 22 provided in the connecting portion 20 and made of the harder PP plastic does not extend over the entire periphery in the connecting portion 20, the flexibility of the connecting portion 20 is nevertheless safeguarded. In addition, the connecting web 22 is injection-molded around by a soft TPE plastic such that the attachment 6 is designed to be closed even in the connecting portion 20, thus reliably enabling the transmission to the mother's breast, of the negative pressure produced by the milk pump 1.

As is apparent from FIG. 9, it is beneficial if the web 22 according to the invention has a ribbed surface such that the elasticity of the connection web 22 will be increased and the cover 23 made of softer TPE plastic will be reliably connected with the connecting web 22.

FIG. 10 depicts yet another exemplary embodiment of the attachment 6, wherein, in this case, a dimensionally stable application portion 7 is again made of a comparatively hard plastic material, in which massage areas 12 of softer plastic are merely provided. The comparatively hard application portion 7 is adjoined by a connecting portion 20 of softer plastic, preferably TPE, having a substantially undulated or bowed structure. The fixing portion 8 may likewise be comprised of softer TPE plastic just as the connecting portion 20 or, however, made of harder plastic just as the application portion 7.

What is, however, essential is that the longitudinal axes 7′ and 8′ of the application and fixing portions 7, 8 are readily pivotable into positions deviating from 180°.

1. An attachment for a milk pump, including a single-walled, substantially dimensionally stable application portion tapering in a funnel-shaped manner from the application opening to a fixing portion provided for connection with the milk pump, wherein the application portion and the fixing portion are interconnected by an articulated connection such that the longitudinal axis of the funnel-shaped application portion is adjustable relative to a longitudinal axis of the fixing portion at an angle deviating from 180°.

2. An attachment according to claim 1, wherein the application portion and the fixing portion are interconnected by a universal-joint connection.

3. An attachment according to claim 1, wherein the application portion and the fixing portion are interconnected by a flexible connecting portion.

4. An attachment according to claim 3, wherein the flexible connecting portion is folded like bellows or corrugated.

5. An attachment according to claim 3, wherein the attachment is made of at least two materials of different hardnesses.

6. An attachment according to claim 5, wherein a thermoplastic elastomer, silicone or rubber is provided as the softer material.

7. An attachment according to claim 5, wherein the softer material has a Shore hardness A of between 20 and 70.

8. An attachment according to claim 5, wherein polypropylene, polycarbonate or another thermoplastic material is provided as the harder material.

9. An attachment according to claim 5, wherein the harder material has a Rockwell hardness R of between 80 and 120.

10. An attachment according to claim 3, wherein the flexible connecting portion comprises at least a further plastic material differing from that of the application portion and the fixing portion.

11. An attachment according to claim 3, wherein at least one web connecting the application portion and the fixing portion is provided in the flexible connecting portion, said web being made of the same plastic as the application portion and the fixing portion.

12. An attachment according to claim 11, wherein a softer plastic is sprayed on the web in the region of the connecting portion.

13. An attachment according to claim 11, wherein the web is ribbed or corrugated.

14. An attachment according to claim 3, wherein the flexible connecting portion is comprised of softer plastic than the application portion and the fixing portion.

15. An attachment according to claim 3, wherein the flexible connecting portion and the fixing portion are comprised of softer plastic than the application portion.

16. An attachment according to claim 3, wherein the attachment comprises struts of the harder material and a cover of the softer material.

17. An attachment according to claim 16, wherein the attachment comprises struts extending in the direction of the longitudinal axis.

18. An attachment according to claim 16, wherein the attachment comprises circular struts arranged perpendicularly to the longitudinal axis in particular, in the region of the application portion.

19. An attachment according to claim 3, wherein the attachment is a one-piece two-component injection molded part.

20. An attachment according to claim 1, wherein the fixing portion is conically designed.

21. An attachment according to characterized in that claim 1, wherein the fixing portion is cylindrically designed.

22. An attachment according to claim 1, wherein the longitudinal axis of the application portion is adjustable relative to the longitudinal axis of the fixing portion at an angle of between 150° and 170°.

23. A milk pump including a pumping device having a suction socket in communication with a funnel-shaped application portion of an attachment to be applied to a mother's breast, wherein the spatial orientation of a longitudinal axis of the funnel-shaped application portion relative to a longitudinal axis of the suction socket is adjustable, wherein an attachment according to claim 1 is provided.
